(19) United States
(12) Patent Application Publication
(10) Pub. No.: US 2010/0203600 A1
(43) Pub. Date: Aug. 12, 2010

(54) METHOD FOR THE COPRODUCTION OF METHYL 7-OXOHEPTANOATE AND UNDECYLENIC ACID FROM RICINOLEIC ACID

(75) Inventor: Jean-Luc Dubois, Millery (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 12/678,617

(22) PCT Filed: Sep. 17, 2008
(86) PCT No.: PCT/FR08/51666

§ 371 Date: Mar. 17, 2010

(30) Foreign Application Priority Data
Sep. 20, 2007 (FR) 0757696

(51) Int. Cl. C12P 13/00; C12P 7/64

(52) U.S. Cl. 435/128; 435/134

(57) ABSTRACT

The invention relates to a method for converting ricinoleic acid that comprises the following successive stages:
a) fermentation of ricinoleic acid by a bacterium, a yeast or a fungus, in order to obtain 7-hydroxy-9-octadecenedioic diacid,
b) the esterification of the 7-hydroxy-9-octadecenedioic diacid obtained in-stage during step a), in order to form the corresponding methyl diester,
c) high-temperature cracking of the product obtained during step b), in order to form a mixture of methyl undecylenate and methyl 7-oxoheptanoate,
d) separating the methyl undecylenate and methyl 7-oxoheptanoate obtained in step c),
e) hydrolysing the methyl undecylenate obtained in step d), for forming the undecylenic acid.

The invention is targeted at a process for the conversion of ricinoleic acid including a stage of fermentation of this acid, for the purpose of obtaining in particular methyl 7-oxoheptanoate and 10-undecylenic acid (also known as undecyl-10-enoic acid and subsequently known more simply as undecylenic acid).

The fermentation of ricinoleic acid results in the diacid of nonadecene.

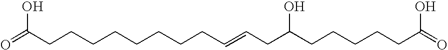

The most well known unsaturated diacids or diesters are those comprising chains comprising from 4 to 6 carbon atoms, such as the C₄ acids maleic acid and fumaric acid, the C₅ acids citraconic acid, mesaconic acid and itaconic acid and the C₆ acids 2-methyleneglutaric acid and muconic acid. On the other hand, as regards long-chain diacids, the only ones having a degree of importance are dimers generally obtained by condensation of unsaturated carboxylic acids. The properties, syntheses and uses of these diacids are described in the Kirk-Othmer Encyclopedia, Vol. A8, pages 533-536.

Saturated diacids are obtained industrially by various methods, all of which, however, exhibit some disadvantages. A great variety of these methods is enlarged upon in the above reference on pages 523-536.

It is possible to distinguish therein methods by degradation, such as ozonolysis or oxidation, of vegetable fatty acids.

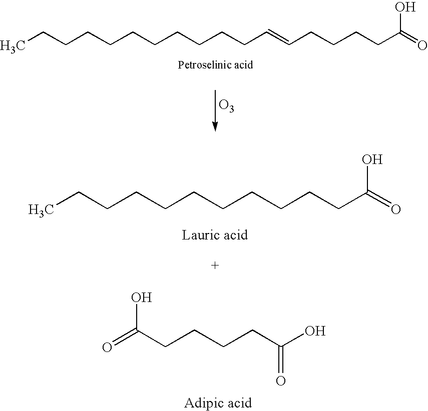

The ozonolysis of oleic acid, of petroselinic acid and of erucic acid makes it possible to respectively produce the diacids comprising 9, 6 and 13 carbon atoms according to the above reaction process for petroselinic acid.

Another example is the cleavage of ricinoleic acid by the action of sodium hydroxide at a temperature of greater than 180° C. This method, used industrially, makes it possible to obtain the diacid comprising 10 carbon atoms. The same method, as illustrated in the scheme below, can be applied to lesquerolic acid and results in the formation of a diacid comprising 12 carbon in atoms. This method exhibits the advantage of using renewable starting materials but is restricted essentially to the C₁₀ diacid, lesquerolic acid being still not very widespread, and thus this method is relatively little used.

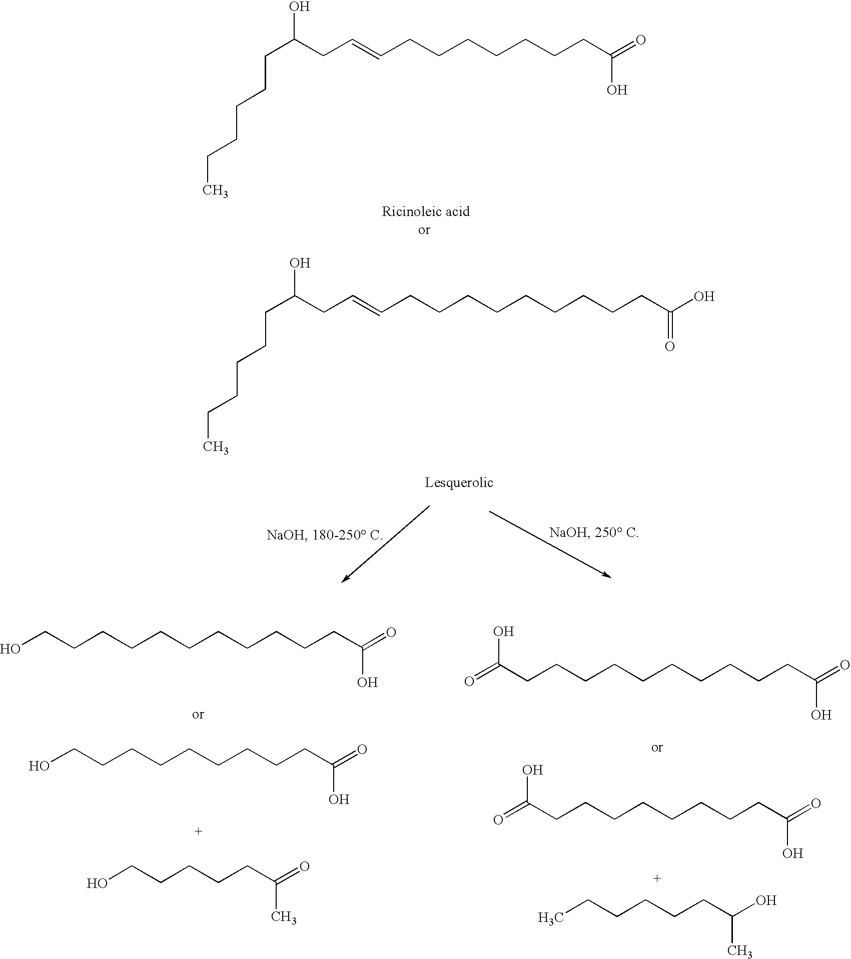

Mention may also be made of the oxidative degradation of monocarboxylic acids by the action of N₂O₄. The oxidation of stearic acid makes it possible to obtain a mixture of sebacic acid and of caprylic acid; suberic acid can be obtained from palmitic acid.

It is also possible to obtain diacids from smaller molecules by using variant techniques of carbonylation.

Finally, mention may be made of fermentation, by yeast, a fungus or a bacterium, of saturated or unsaturated fatty ester, acid, alcohol or paraffin hydrocarbon substrates, which makes it possible to oxidize the compounds of the substrate. This method is well known. It is illustrated in particular in the paper by W. H. Eschenfeidt et al. “Transformation of Fatty Acids Catalyzed by Cytochrome P450 Monooxygenase Enzymes of Candida tropicalis”, and patents FR 2 445 374, U.S. Pat. No. 4,474,882, U.S. Pat. No. 3,823,070, U.S. Pat. No. 3,912,586, is U.S. Pat. No. 6,660,505, U.S. Pat. Nos. 6,569,670 and 5,254,466. It makes it possible to obtain numerous diacids of variable chain length. The production of 9-octadecenedioic diacid by fermentation of oleic acid and oleyl alcohol is described in the publication by Zu-Hua Yi and Hans-Jürgen Rehm in Appl. Microbiol. Biotechnol., (1989), 30, 327-331.

The literature is relatively poor in documents illustrating the formation of hydroxylated unsaturated diacids. The publication by D. Fabritius, H. J. Schafer and A. Steinbüchel in Appl. Microbiol. Biotechnol., (1996), 45, 342-348, illustrates the formation of 3-hydroxy-9-octadecenedioic acid by fermentation of oleic acid with a mutant strain of Candida Tropicalis. The mutation of the C. tropicalis DSM 3152 strain with N-methyl-N-nitro-N′-nitrosoguanidine resulted in a mutant M25 which makes it possible to obtain a concentration of hydroxylated diacid 1.8 times higher than with the mother strain.

U.S. Pat. No. 6,569,670 describes a fermentation process for the production of diacids. The use of natural fatty acids is described in column 6, line 25. in particular, ricinoleic acid, its esters and triglycerides having a high content of ricinoleic acid are described, although not illustrated.

Current developments with regard to the environment are resulting in the use of natural starting materials originating from a renewable source being favored in the fields of energy and chemistry. Thus it is that fatty acids/esters extracted from oils or fats of vegetable or animal origin, which are thus renewable, are becoming particularly advantageous starting materials.

An example of an industrial process using a fatty acid as starting material is that of the manufacture, starting from ricinoleic acid extracted from castor oil, of 11-aminoundecanoic acid, which forms the basis of the Rilsan® synthesis. This process is described in the work “Les Procédés de Pétrochimie” [Petrochemical Processes] by A. Chauvel et al., which appeared in Editions TECHNIP (1986). 11-Aminoundecanoic acid is obtained in several stages. The first consists of a methanolysis of castor oil in a basic medium, producing methyl ricinoleate, which is subsequently subjected to a pyrolysis in order to obtain, on the one hand, heptanaldehyde and, on the other hand, methyl undecylenate. The latter is converted to the acid form by hydrolysis. Subsequently, the acid formed is subjected to a hydrobromination to give the ω-brominated acid, which is converted by amination to 11-aminoundecanoic acid.

This process results in the coproduction of 11-aminoundecanoic acid and heptanaldehyde, which should be recovered in value separately. Heptanaldehyde has applications in perfumery or fine chemistry markets, or also has to be converted to heptanoic acid by oxidation, for applications in oils, plasticizers or paints, or also has to be converted to heptanol, for applications in plasticizers, health and safety. Furthermore, heptanaldehyde has only a limited market and is mainly converted, in particular into heptanoic acid, to be recovered in value.

Castor oil comprises approximately 85% of ricinoleic acid. In the conventional process for the production of 10-undecylenic acid, the other fatty acids present in castor oil are not cracked and result in a fraction, known as Esterol, comprising compounds having a low added value.

These markets can be highly fluctuating and it is desirable to find a process which can make it possible to avoid the production of heptanaldehyde while promoting the formation of other compounds which can be used industrially, for example in the production of industrial polymers or in the pharmaceutical industry.

In the pharmaceutical industry, methyl 7-oxoheptanoate is of particular use; in particular, during the synthesis of prostanoids, such as 2-(6-methoxycarbonylhexyl)cyclopent-2-en-1-one (Bosone, Enrico; Synthesis 1983 (11), pp 942-4), or during the synthesis of prostaglandins, such as Mexiprostil®, it is necessary to have available methyl 7-oxoheptanoate (Kolb, M., Tetrahedron Letters, 1988, Vol. 29 (51), pp 6769-72).

Moreover, undecylenic acid is also of use in the field of the pharmaceutical industry, where it is used as active principle in compositions intended for the treatment of mycoses.

It is thus necessary to find a type of process which makes it possible to obtain both methyl 7-oxoheptanoate and undecylenic acid and which in addition uses a renewable material of natural origin, castor oil.

A subject matter of the invention is thus a process for the conversion of castor oil.

According to the invention, said process comprises the following successive stages:
a) the fermentation of ricinoleic acid by a bacterium, a yeast or a fungus, in s order to obtain 7-hydroxy-9-octadecenedioic diacid,
b) the esterification of the 7-hydroxy-9-octadecenedioic diacid obtained in stage a), in order to form the corresponding methyl diester,
c) the high-temperature cracking of the product obtained in stage b), in order to form a mixture of methyl undecylenate and methyl 7-oxoheptanoate,
d) the separation of the methyl undecylenate and methyl 7-oxoheptanoate obtained in stage c),
e) the hydrolysis of the methyl undecylenate obtained in stage d), in order to form undecylenic acid.

According to the process of the invention, methyl 7-oxoheptanoate is obtained on completion of stage d), and undecylenic acid is obtained on conclusion of stage e).

In preferred embodiments of the invention, the undecylenic acid obtained in stage e) can be converted to 11-aminoundecanoic acid and the methyl 7-oxoheptanoate obtained in stage d) can be converted to 7-aminoheptanoic acid.

These compounds, which are amino acids, are of particular use in the chemical industry and in particular the polymer industry, such as the production of polyamides or of industrial polymers.

Consequently, the process which is a subject matter of the invention can additionally comprise the following stages:
f) the reaction of the undecylenic acid obtained in stage e) with HBr, in order to form 11-bromoundecylenic acid,
g) the reaction of the 11-bromoundecylenic acid obtained in stage f) with ammonia, in order to form 11-aminoundecanoic acid.

The process which is the subject matter of the invention can also comprise a stage of reductive amination of the methyl 7-oxoheptanoate obtained in stage d), followed by a hydrolysis stage, in order to form 7-aminoheptanoic acid.

7-Aminoheptanoic acid is a remarkable monomer. This is because this monomer has little tendency to cyclize and polyamide 7, which results from 7-aminoheptanoic acid, has a higher melting point than polyamide 6.

Preferably, the ricinoleic acid results from vegetable oils mainly extracted from the castor oil plant but also from some algae and from genetically modified or unmodified plants, such as flax or rape.

The ricinoleic acid employed in stage a) can originate from castor oil. Castor oil comprises approximately 85% of ricinoleic acid and a small percentage of other fatty acids.

When castor oil is employed in stage a) of the process defined above, the fatty acids other than the ricinoleic add present in this oil are converted, after the high-temperature cracking stage c), to C₁₈, C₁₆ and C₂₀ diacids, which themselves have applications in the production of industrial polymers.

In the continuation of the present description, the term “acid” will be used, for the clarity of the account, to denote without distinction the acid and the ester or the triglyceride. This is because, in the process of the invention, ricinoleic acid can be treated either in its acid form or in its ester form or in its triglyceride form. The change from one form to the other is carried out by alcoholysis, esterification, transesterification or hydrolysis.

In the process of the invention, use is made of fatty acids or esters of natural origin, that is to say present in oils or fats. The latter are in fact composed, apart from the ester or acid participating in the reaction, of a mixture of esters or acids of closely related formulae. By way of example, castor oil comprises, in addition to ricinoleic acid, simultaneously oleic acid and linoleic acid, palmitic acid, stearic acid and arachidic acid. The presence of these saturated, monounsaturated, diunsaturated or polyunsaturated acids has no significant consequences with regard to the progress of the process insofar as, during the cracking stage, separation of the products is carried out.

The first stage of the process which is a subject matter of the invention, stage a), is carried out by fermentation in the presence of a microorganism, that is to say using any bacterium, fungus or yeast which makes possible oxidation of the fatty ester, triglyceride or acid of the feedstock.

The fermentation stage can be carried out in the presence of a microorganism comprising an enzyme which makes it possible to obtain 7-hydroxy-9-octadecenedioic diacid.

It is possible without distinction to ferment a fatty acid, a fatty ester and a triglyceride. This is because the microorganisms are also capable of metabolizing alcohols and glycerol.

Stage b) of the process which is a subject matter of the invention consists of the esterification of the 7-hydroxy-9-octadecenedioic diacid obtained o in stage a), in order to form the corresponding methyl diester. Preferably, this esterification stage takes place in the presence of an excess of methanol and of a catalyst, for example an acid catalyst.

Stage c) of high-temperature cracking of the product obtained in stage b), in order to form a mixture of methyl undecylenate and methyl 7-oxoheptanoate, is preferably takes place at a temperature ranging from 400° C. to 600° C.

The separation stage d) is a stage conventional for a person skilled in the art and will thus not be described in detail. By way of example, the separation can consist of a distillation.

The hydrolysis stage e) preferably takes place at a temperature close to ambient temperature, in the presence of sodium hydroxide, but can also be carried out at 80° C. and under pressure, for example.

Stage f) consists of a hydrobromination, preferably carried out in the presence of a solvent, for example toluene, and of a free radical initiator, such as benzoyl peroxide. Finally, the amination stage g) preferably takes place in the presence of a large excess of an aqueous ammonia solution.

The reductive amination of the 7-oxoheptanoic acid obtained in stage c), in order to form 7-aminoheptanoic acid, can be carried out according to numerous catalytic or enzymatic methods, for example according to the method described in patent application U.S. Pat. No. 5,973,208.

In one embodiment of the process which is a subject matter of the invention, lesquerolic acid is used as a replacement for ricinoleic acid.

Thus, the invention also relates to a process for the conversion of lesquerolic acid comprising the following successive stages:
a) the fermentation of lesquerolic acid by a bacterium, a yeast or a fungus, in order to obtain 7-hydroxy-11-eicosenedioic diacid,
b) the esterification of the 7-hydroxy-11-eicosenedioic diacid obtained in stage a), in order to form the corresponding methyl diester,
c) the high-temperature cracking of the product obtained in stage b), in order to form a mixture of methyl 12-tridecenoate and methyl 7-oxoheptanoate,
d) the separation of the methyl 12-tridecenoate and methyl 7-oxoheptanoate obtained in stage c),
e) the hydrolysis of the methyl 12-tridecenoate obtained in stage d), in order to form 12-tridecenoic acid.

This process can additionally comprise a stage of reductive amination of the methyl 7-oxoheptanoate obtained in stage d), followed by a hydrolysis is stage, in order to form 7-aminoheptanoic acid.

The following examples illustrate, without limiting it, the process which is a subject matter of the invention, employing ricinoleic acid.

### Examples

### Example 1

### Stage a) of the Process, Fermentation of Ricinoleic Acid

In this example, a yeast strain is cultured in a medium comprising ricinoleic acid. The mixture is subsequently sterilized at 120° C. for 15 minutes. The culture is maintained at 30° C. A sodium hydroxide solution is added continuously in order to maintain the pH from 7 to −7.5. After culturing for 48 hours, the hydroxylated unsaturated diacid is recovered by extraction with diethyl ether. It is possible, by employing this procedure, to obtain 7-hydroxy-9-octadecenedioic diacid.

### Example 2

### Stage b) of the process, esterification of 7-hydroxy-9-octa-decenedioic diacid

The diacid produced is esterified to give the methyl diester by reaction in the presence of an acid catalyst and of methanol. 1 g of diacid is brought together with methanol in the presence of a sulfated zirconia heterogeneous acid catalyst obtained from Wako. The reaction mixture is heated at 150° C. until equilibrium is reached and then the excess methanol is distilled off. These s experimental conditions make it possible to obtain the methyl diester of 7-hydroxy-9-octadecenedioic diacid.

### Example 3

### Stage c) of the Process, Thermal Cracking of the Diester

Thermal cracking of the methyl diester is carried out at 500° C. in a tubular furnace with a residence time of 15 seconds. After vaporization of the diester at 260° C., it is mixed with steam heated to 600° C. The stream resulting from this reaction is first of all condensed in a series of exchangers where the temperature is lowered to approximately 150° C. The water is then separated by settling. The products formed are fractionated in successive distillation columns. With these experimental conditions, it is possible to thus recover the fractions of the methyl ester of undecylenic acid, the ester of 7-oxoheptanoic acid and the diesters other than that resulting from ricinoleic acid. The unconverted product can be recycled at the inlet of the reactor after separation.

Stages d), e) and f) of the process which is a subject matter of the invention are stages conventional for a person skilled in the art and are therefore not illustrated.

### Example 4

### Reductive Amination of the Methyl Ester of 7-oxoheptanoic Acid

1 g of copper chromite catalyst, 8 g of the methyl ester of 7-oxoheptanoic acid, 30 g of ethanol and 30 g of ammonia are introduced into an autoclave equipped with a magnetic stirrer. Hydrogen is subsequently introduced at a pressure of 80 bar. The autoclave is subsequently heated to 150° C. over 1 hour and maintained at this temperature for 2 hours. After filtering, in order to remove the catalyst, and removing the solvent, the product is analyzed. These experimental conditions make it possible to obtain the methyl ester of 7-aminoheptanoic acid. The latter can subsequently be hydrolyzed in order to form 7-aminoheptanoic acid. The hydrolysis is carried out at 25° C. in a jacketed reactor with a residence time of 30 minutes. The methanol can be recycled after purification to the esterification stage.

1. A process for the conversion of ricinoleic acid comprising the following successive steps:
a) fermenting ricinoleic acid by a bacterium, a yeast or a fungus, to obtain 7-hydroxy-9-octadecenedioic diacid,
b) the esterification of the 7-hydroxy-9-octadecenedioic diacid obtained in step a), to form the corresponding methyl diester,
c) high-temperature cracking of the product obtained in step b), to form a mixture of methyl undecylenate and methyl 7-oxoheptanoate,
d) separation of the methyl undecylenate and methyl 7-oxoheptanoate obtained in step c),
e) hydrolysis of the methyl undecylenate obtained in stage step d), to form undecylenic acid.

2. The process as claimed in claim 1, characterized in that it additionally comprises the following steps:
f) reaction of the undecylenic acid obtained in step e) with HBr, to form 11-bromoundecylenic acid,
g) the reaction of the 11-bromoundecylenic acid obtained in stage f) with ammonia, to form 11-aminoundecanoic acid.

3. The process as claimed in claim 1, characterized in that it further comprises a step of reductive amination of the methyl 7-oxoheptanoate obtained in step d), followed by a hydrolysis step, to form 7-aminoheptanoic acid.
